(19) United States
(12) Patent Application Publication
(10) Pub. No.: US 2011/0098453 A1
(43) Pub. Date: Apr. 28, 2011

(54) MAGNETIC NANOCOMPOSITE, AND PROCESS FOR SELECTIVE BINDING, SEPARATION AND PURIFICATION OF PROTEIN USING THE SAME

(75) Inventors: Taeghwan Hyeon, Seoul (KR); Jae-Yoon Kim, Namyangju-si (KR); Nohyun Lee, Seoul (KR); Yuanzhe Piao, Seoul (KR)

(73) Assignee: SNU R&DB FOUNDATION, Seoul (KR)

(21) Appl. No.: 12/852,774

(22) Filed: Aug. 9, 2010

(30) Foreign Application Priority Data
Aug. 10, 2009 (KR) 10-2009-0073453

(51) Int. Cl. C07K 1/14; B05D 5/12; H01F 1/00; H01F 1/01; B82Y 5/00

(52) U.S. Cl. 530/415; 427/127; 252/62.51 R; 252/62.56; 252/62.51 C; 977/773

(57) ABSTRACT

The present invention relates to a magnetic nanocomposite, a process for production thereof, a reusable protein-binding agent for separation of a protein including the magnetic nanocomposite, and a process for selective binding, separation and purification of a protein using the magnetic nanocomposite. In particular, the present invention is directed to a magnetic nanocomposite with a magnetic nanoparticle core of a magnetic nanoparticle, a silica shell coating said core, and a nanoparticle layer of a fourth period transition metal oxide, which coats said silica shell, a process for production of the magnetic nanocomposite, a reusable protein-binding agent the magnetic nanocomposite, and a process for selective binding, separation and purification of a protein using the magnetic nanocomposite.

### TECHNICAL FIELD

The present invention relates to a magnetic nanocomposite, a process for production thereof, a reusable protein-binding agent for separation of a protein including the magnetic nanocomposite, and a process for selective binding, separation and purification of a protein using the magnetic nanocomposite. In particular, the present invention is directed to a magnetic nanocomposite comprising a magnetic nanoparticle core of a magnetic nanoparticle, a silica shell coating said core, and a nanoparticle layer of a fourth period transition metal oxide, which coats said silica shell, a process for production of the magnetic nanocomposite, a reusable protein-binding agent the magnetic nanocomposite, and a process for selective binding, separation and purification of a protein using the magnetic nanocomposite.

### BACKGROUND OF THE INVENTION

Easy separation and manipulation of recombinant proteins are very important in proteomics. Nickel nitrilotriacetic acid (Ni-NTA) beads, the most frequently used for separating recombinant proteins, have been used to purify proteins with the oligohistidine affinity tag (His-tag). The Ni-NTA bead employs an NTA-attached polymer resin to immobilize nickel ions (Ni²⁺) and thereby separates His-tagged proteins through coordination chemistry.

However, conventional Ni-NTA system has some limitations including pretreatment to remove cell debris and colloid contaminants, a relatively long operation time, solvent consumption, and protein solubility.

Magnetic nanoparticles, recently, have been extensively researched for various biomedical applications including magnetic resonance imaging contrast agent, magnetic targeting of drug delivery vehicles, and magnetic separation of biomolecules of DNA, proteins, and cells. Recently, several magnetic separation systems based on magnetic nanomaterials have been reported to circumvent the limitations of Ni-NTA system mentioned above.

Xu et al. synthesized NTA-modified magnetic nanocrystals of FePt and Co/Fe₂O₃ and demonstrated their separation properties of His-tagged proteins (C. Xu, K. Xu, H. Gu, X. Zhong, Z. Guo, R. Zheng, X. Zhang, B. Xu, J. Am. Chem. Soc. 2004, 126, 3392; C. Xu, K. Xu, H. Gu, R. Zheng, H. Liu, X. Zhang, Z. Guo, B. Xu, J. Am. Chem. Soc. 2004, 126, 9938).

Mirkin et al. fabricated Au—Ni—Au triblock nanorods using anodic alumina membrane and applied them to the magnetic separation of His-tagged proteins (K.-B. Lee, S. Park, C. A. Mirkin, Angew. Chem. 2004, 116, 3110; Angew. Chem. Int. Ed. 2004, 43, 3048; B.-K. Oh, S. Park, J. E. Millstone, S. W. Lee, K.-B. Lee, C. A. Mirkin, J. Am. Chem. Soc. 2006, 128, 11825).

More recently, Hyeon et al. reported Ni/NiO core/shell nanoparticles for selective binding and magnetic separation of His-tagged proteins (I. S. Lee, N. Lee, J. Park, B. H. Kim, Y.-W. Yi, T. Kim, T. K. Kim, I. H. Lee, S. R. Paik, T. Hyeon, J. Am. Chem. Soc. 2006, 128, 10658).

However, these systems have been suffered from the weak magnetic properties and subsequent low recyclability. For example, when we used Ni/NiO nanoparticles several times, the magnetic properties of Ni/NiO core/shell nanoparticles gradually decreased because the magnetic Ni cores gradually oxidized to antiferromagnetic NiO.

To address this recycling problem, herein, the present inventors demonstrate on a novel magnetically recyclable (reusable) protein separation system using magnetic nanocomposites for purifying, for example, His-tagged proteins from a protein mixture solution and from cell lysates.

### TECHNICAL PROBLEM

The primary object of the present invention is to provide a magnetic nanocomposite comprising a magnetic nanoparticle core of a magnetic nanoparticle, a silica shell coating said core, and a nanoparticle layer of a fourth period transition metal oxide, which coats said silica shell.

Another object of the present invention is to provide a process for production of a magnetic nanocomposite, comprising: (i) reacting a magnetic nanoparticle with a silica precursor to form a silica shell on said magnetic nanoparticle; (ii) introducing a salt or ion of a fourth period transition metal to said silica shell; and (iii) heating the particle formed in said step (ii) to form a nanoparticle layer of a fourth period transition metal oxide, which coats said silica shell.

Yet another object of the present invention is to provide a reusable protein-binding agent which comprises a magnetic nanocomposite comprising a magnetic nanoparticle core of a magnetic nanoparticle, a silica shell coating said core, and a nanoparticle layer of a fourth period transition metal oxide, which coats said silica shell, said reusable protein-binding agent selectively binding a protein including an amino acid selected from the group consisting of histidine, asparagine, arginine, cystine, glutamine, lysine, methionine, proline and tryptophan.

Still another object of the present invention is to provide a process for selective binding, separation and purification of a protein, comprising: (i) binding a protein-binding agent comprising a magnetic nanocomposite comprising a magnetic nanoparticle core of a magnetic nanoparticle, a silica shell coating said core, and a nanoparticle layer of a fourth period transition metal oxide, which coats said silica shell, with said protein contained in a protein mixture solution or cell lysate; (ii) separating said protein bound with said protein-binding agent from said protein mixture solution or cell lysate by an external magnetic field; and (iii) isolating said protein bound with said separated protein-binding agent from said magnetic nanocomposite.

### DETAILED DESCRIPTION OF THE INVENTION

The above-mentioned primary object of the present invention can be achieved by providing a magnetic nanocomposite comprising a magnetic nanoparticle core of a magnetic nanoparticle, a silica shell coating said core, and a nanoparticle layer of a fourth period transition metal oxide, which coats said silica shell.

The magnetic nanoparticle which constitutes the core of the magnetic nanocomposite, may be selected from transition metal oxides, transition metal phosphides, transition metal sulfides or transition metal alloys which are ferromagnetic or superparamagnetic. Preferably, the transition metal may be selected from iron (Fe), manganese (Mn), chromium (Cr), cobalt (Co), nickel (Ni), copper (Cu), zinc (Zn), samarium (Sm), gadolinium (Gd), neodymium (Nd), europium (Eu), barium (Ba) or platinum (Pt). In addition, the magnetic nanoparticle core preferably has a diameter ranging from 1 nm to 1,000 nm.

The core has magnetic properties and, therefore, endows the magnetic nanocomposite of the present invention with magnetism. Moreover, ions on the surface of the fourth period transition metal oxide nanoparticles make it possible that the magnetic composite of the present invention binds an amino acid such as histidine.

The silica which constitutes the shell of the magnetic nanocomposite of the present invention may be crystalline, noncrystalline or porous. Preferably, the silica shell has a thickness ranging from 1 nm to 1,000 nm.

Preferably, the fourth period transition metal oxide which constitutes the nanoparticle layer of the magnetic nanocomposite of the present invention is selected from an oxide of chromium (Cr), manganese (Mn), iron (Fe), cobalt (Co), nickel (Ni), copper (Cu) or zinc (Zn).

Preferably, the fourth period transition metal oxide nanoparticle has a diameter ranging from 1 nm to 100 nm and the nanoparticle layer of a fourth period transition metal oxide has a thickness ranging from 1 nm to 1,000 nm.

Another object of the present invention can be achieved by providing a process for production of a magnetic nanocomposite, comprising: (i) reacting a magnetic nanoparticle with a silica precursor to form a silica shell on said magnetic nanoparticle; (ii) introducing a salt or ion of a fourth period transition metal to said silica shell; and (iii) heating the particle formed in said step (ii) to form a nanoparticle layer of a fourth period transition metal oxide, which coats said silica shell.

The magnetic nanoparticle in the step (i) of the process for production of a magnetic nanocomposite of the present invention may be selected from transition metal oxides, transition metal phosphides, transition metal sulfides or transition metal alloys which are ferromagnetic or superparamagnetic. Preferably, the transition metal may be selected from iron (Fe), manganese (Mn), chromium (Cr), cobalt (Co), nickel (Ni), copper (Cu), zinc (Zn), samarium (Sm), gadolinium (Gd), neodymium (Nd), europium (Eu), barium (Ba) or platinum (Pt). In addition, the magnetic nanoparticle core preferably has a diameter ranging from 1 nm to 1,000 nm.

Preferably, the silica precursor which is used in the process for production of a magnetic nanocomposite of the present invention is selected from tetraethyl orthosilicate (TEOS, Si(OC₂H₅)₄), tetramethyl orthosilicate (TMOS, Si(OCH₃)₄) or silicon tetrachloride (SiCl₄). In addition, the silica shell formed in the step (i) preferably has a thickness ranging from 1 nm to 1,000 nm.

Preferably, the fourth period transition metal of the step (ii) is selected from chromium (Cr), manganese (Mn), iron (Fe), cobalt (Co), nickel (Ni), copper (Cu) or zinc (Zn).

The procedure of introduction of a fourth period transition metal salt into the silica shell in the step (ii) may be carried out by the following two methods.

Firstly, the fourth period transition metal salt may be introduced into mesopores after the mesopores have been formed outer part of the silica shell.

The mesopores may be formed by reacting the silica shell with a mixture of tetraethyl orthosilicate and n-octadecyltrimethoxysilane. In addition, the fourth period transition metal salt may be introduced into the mesopores by impregnating the fourth period transition metal salt.

Secondly, when a functional group such as amine, carboxyl, thiol and the like is introduced on the surface of the silica shell and is reacted with a fourth period transition metal salt, the fourth period transition metal ion is introduced in the surface of the silica shell by formation of a coordination bond between the fourth period transition metal ion and the functional group.

In order to introduce an amine group on the surface of the silica shell, the silica shell is reacted with a silane derivative including an amine group, such as 3-aminopropyltriethoxysilane, 3-aminopropyltrimethoxysilane, 4-aminobutyltriethoxysilane or 11-aminoundecyltriethoxysilane.

In addition, in order to introduce a carboxyl group on the surface of the silica shell, the silica shell is reacted with a silane derivative including a carboxyl group, such as carboxyethylsilanetriol sodium salt, triethoxysilylpropylmaleamic acid or N-(trimethoxysilylpropyl)ethylene diamine triacetic acid trisodium salt.

Moreover, in order to introduce a thiol group on the surface of the silica shell, the silica shell is reacted with a silane derivative including a thiol group, such as 3-mercaptopropyltrimethoxysilane, 3-mercaptopropyltriethoxysilane or 11-mercaptoundecyltrimethoxysilane.

In the step (iii), the fourth period transition metal oxide nanoparticles are formed when the fourth period transition metal salt introduced into the mesopores formed in the silica shell, or the fourth period transition metal ion coordinated to the functional group introduce on the surface of the silica shell.

Preferably, the step (iii) is carried out under hydrogen and nitrogen atmosphere at temperature of 100° C. to 1,000° C. for 10 min to 12 hr.

In addition, the fourth period transition metal oxide preferably has a diameter ranging from 1 nm to 100 nm, and the nanoparticle layer of the fourth period transition metal oxide preferably has a thickness ranging from 1 nm to 1,000 nm.

Yet another object of the present invention can be achieved by providing a reusable protein-binding agent which comprises a magnetic nanocomposite comprising a magnetic nanoparticle core of a magnetic nanoparticle, a silica shell coating said core, and a nanoparticle layer of a fourth period transition metal oxide, which coats said silica shell, said reusable protein-binding agent selectively binding a protein including an amino acid selected from the group consisting of histidine, asparagine, arginine, cystine, glutamine, lysine, methionine, proline and tryptophan.

The magnetic nanoparticle which constitutes the core of the protein-binding agent of the present invention may be selected from transition metal oxides, transition metal phosphides, transition metal sulfides or transition metal alloys which are ferromagnetic or superparamagnetic. Preferably, the transition metal may be selected from iron (Fe), manganese (Mn), chromium (Cr), cobalt (Co), nickel (Ni), copper (Cu), zinc (Zn), samarium (Sm), gadolinium (Gd), neodymium (Nd), europium (Eu), barium (Ba) or platinum (Pt). In addition, the magnetic nanoparticle core preferably has a diameter ranging from 1 nm to 1,000 nm.

The core has magnetic properties and, therefore, endows the protein-binding agent of the present invention with strong magnetism. Moreover, ions on the surface of the fourth period transition metal oxide nanoparticles make it possible that the protein-binding agent of the present invention binds an amino acid such as histidine.

The silica which constitutes the shell of the protein-binding agent of the present invention may be crystalline, noncrystalline or porous. Preferably, the silica shell has a thickness ranging from 1 nm to 1,000 nm.

Preferably, the fourth period transition metal oxide which constitutes the nanoparticle layer of the protein-binding agent of the present invention is selected from an oxide of chromium (Cr), manganese (Mn), iron (Fe), cobalt (Co), nickel (Ni), copper (Cu) or zinc (Zn).

Preferably, the fourth period transition metal oxide nanoparticle has a diameter ranging from 1 nm to 100 nm and the nanoparticle layer of a fourth period transition metal oxide has a thickness ranging from 1 nm to 1,000 nm.

Still another object of the present invention can be achieved by providing a process for selective binding, separation and purification of a protein, comprising: (i) binding a protein-binding agent comprising a magnetic nanocomposite comprising a magnetic nanoparticle core of a magnetic nanoparticle, a silica shell coating said core, and a nanoparticle layer of a fourth period transition metal oxide, which coats said silica shell, with said protein contained in a protein mixture solution or cell lysate; (ii) separating said protein bound with said protein-binding agent from said protein mixture solution or cell lysate by an external magnetic field; and (iii) isolating said protein bound with said separated protein-binding agent from said magnetic nanocomposite.

The magnetic nanoparticle which constitutes the core of the protein-binding agent, which is used for the process for selective binding, separation and purification of a protein of the present invention, may be selected from transition metal oxides, transition metal phosphides, transition metal sulfides or transition metal alloys which are ferromagnetic or superparamagnetic. In addition, the magnetic nanoparticle core preferably has a diameter ranging from 1 nm to 1,000 nm.

The core has magnetic properties and, therefore, endows the protein-binding agent of the present invention with magnetism. Moreover, ions on the surface of the fourth period transition metal oxide nanoparticles make it possible that the protein-binding agent of the present invention binds an amino acid such as histidine.

Preferably, the transition metal may be selected from iron (Fe), manganese (Mn), chromium (Cr), cobalt (Co), nickel (Ni), copper (Cu), zinc (Zn), samarium (Sm), gadolinium (Gd), neodymium (Nd), europium (Eu), barium (Ba) or platinum (Pt).

The silica which constitutes the shell of the protein-binding agent of the present invention may be crystalline, noncrystalline or porous and the silica shell preferably has a thickness ranging from 1 nm to 1,000 nm.

Preferably, the fourth period transition metal oxide nanoparticle which constitutes the nanoparticle layer of the protein-binding agent of the present invention is selected from an oxide of chromium (Cr), manganese (Mn), iron (Fe), cobalt (Co), nickel (Ni), copper (Cu) or zinc (Zn).

Preferably, the fourth period transition metal oxide nanoparticle has a diameter ranging from 1 nm to 100 nm and the nanoparticle layer of a fourth period transition metal oxide has a thickness ranging from 1 nm to 1,000 nm.

Proteins that can be separated by the process for selective binding, separation and purification of a protein of the present invention may be a protein containing at least one selected from histidine, asparagine, arginine, cystine, glutamine, lysine, methionine, proline or tryptophan and, preferably, a protein containing histidine.

### ADVANTAGEOUS EFFECTS

The protein-binding agent of the present invention not only enhances an protein separation efficiency but also is recyclably used, by overcoming low magnetic moment and non-recyclability of the conventional protein-binding agents.

### BRIEF DESCRIPTION OF THE DRAWINGS

FIG. 1 shows a) and b) TEM (transmission electron microscope) images of the magnetic nanocomposites of Example 1 of the present invention, c) the pore size distribution of the mesoporous silica shell, and d) the field-dependent magnetization of the magnetic nanocomposite at 300 K.

FIG. 2a shows an image of a solution containing His-tagged GFP and untagged IgG labeled with Cy5 before addition of the magnetic nanocomposite (left, yellow solution, under UV), an image of a solution after incubation with the magnetic nanocomposite and subsequent magnetic separation which represents that His-tagged GFP were removed from the mixture solution by the magnetic nanocomposite (middle, red solution, under UV), and an image of a solution obtained after release of the His-tagged GFP in imidazole solution (right, green solution, under UV); FIG. 2b shows a fluorescent microscopy image of the magnetic nanocomposite obtained after magnetic separation from the mixture solution under GFP filter; FIG. 2c shows a fluorescent microscopy image of the magnetic nanocomposite obtained after magnetic separation from the mixture solution under Cy5 filter; and FIG. 2d is fluorescece spectra showing the change of emission intensity before and after treating the solutions of His-tagged GFP (green) and Cy5-labeled IgG (red) with the magnetic nanocomposite.

FIG. 3a indicates magnetic separation and recycling of the magnetic nanocomposite for selective separation of His-tagged GFP from the mixture protein solution; and FIG. 3b represents SDS-PAGE analysis of E. coli cell lysate containing His-tagged α-synuclein (lane L) and proteins released from reused magnetic nanocomposites up to 4 times (lanes 1^{st}, 2^{nd}, 3^{rd} and 4^{th}). Lane M is molecular weight markers and lane S is α-synuclein without His-tag.

FIG. 4 shows the schematic presentation of the magnetically recyclable (reusable) protein-binding agent of the present invention, which is used for selective binding, separation and purification of a protein.

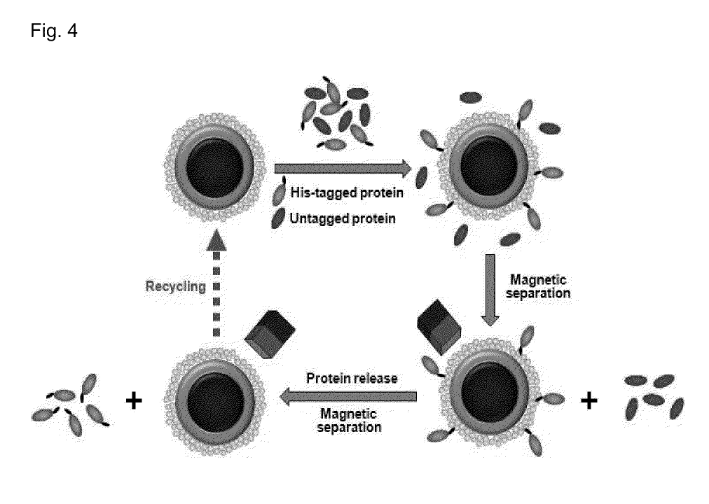

### BEST MODES FOR CARRYING OUT THE INVENTION

Hereinafter, the present invention will be described in greater detail with reference to the following examples and drawings. The examples and drawings are given only for illustration of the present invention and not to be limiting the present invention.

### Example 1

### Synthesis of Magnetic Nanocomposites

0.1 g of hematite particles were stabilized by stirring in 80 mL of water containing 2 g of poly(vinylpyrrolidone) (PVP) for 12 hr. The PVP stabilized hematite nanoparticles were isolated by centrifugation. Then, the PVP stabilized hematite particles were added to a solution containing 90 mL of ethanol, 9 mL of distilled water, and 4 mL of aqueous ammonia solution (30 wt %) at room temperature and the resulting solution was vigorously stirred for 10 min. 0.4 mL of tetraethylorthosilacate (TEOS) was then rapidly added and the solution was vigorously stirred at room temperature for 12 hr to get dense silica shell on the hematite particles.

After being separated by centrifugation, the dense silica coated hematite particles were dispersed in a mixture of 90 mL of ethanol, 9 mL of water and 4 mL of aqueous ammonia solution (30 wt %). The mesoporous silica shell was formed by the simultaneous sol-gel polymerization of a mixture of TEOS and n-octadecyltrimethoxysilane (C₁₈TMS) with a molar ratio of 2:1 for 12 hr at room temperature. The nanocomposite was then calcined at 500° C. for 5 hr to remove the organic groups.

Finally, nickel acetate (weight ratio: 1:1) was introduced into the mesopores of the mesoporous silica shell by impregnation and was then reduced in a flowing mixture of H₂ and N₂ (1:1) at 500° C. for 5 hr to produce Ni nanoparticles. The color of the powder changed from red to deep black after the reduction process, thereby indicating the reduction of the hematite cores to magnetite. The Ni nanoparticles on the MNS were oxidized by exposing them under ambient conditions for 2 days prior to use for separation of His-tagged proteins.

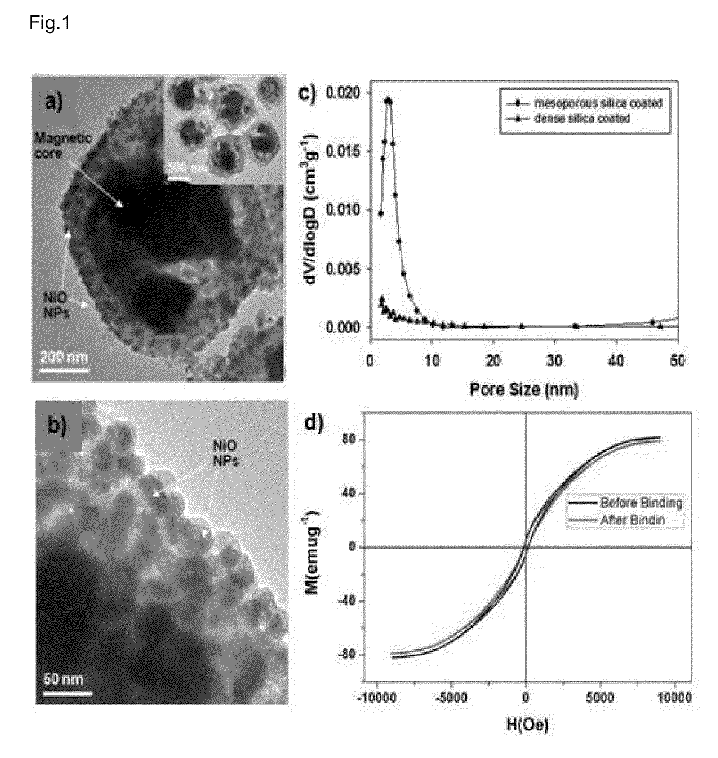

FIG. 1 shows TEM (transmission electron microscope) images (FIGS. 1a and 1b) of the magnetic nanocomposites of Example 1 of the present invention, the pore size distribution of the mesoporous silica shell (FIG. 1c), and the field-dependent magnetization of the magnetic nanocomposite at 300 K (FIG. 1d).

The mesopores generated from the removal of organic groups of C₁₈TMS were observed in the outer mesoporous silica shell. The pore size distribution obtained from the analysis of the adsorption branch using the BJH (Barett-Joyner-Halenda) method demonstrates that the outer silica shell has well-developed mesopores with a diameter of 2.9 nm but hematite nanoparticles coated with only dense silica shells have no clear mesopores (FIG. 1c).

The BET (Brunauer-Emmett-Teller) surface area and the total pore volume of the outer silica shell were measured to be 61 m²/g and 0.27 cm³/g, respectively, while hematite particles coated with dense silica shells had 12 m²/g and 0.07 cm³/g. This higher surface areas and larger accessible pore volumes of the outer silica shell, compared to dense silica coating, make it possible to load higher amount of Ni²⁺ salts into the mesoporous silica shells.

After reduction of Ni²⁺-loaded outer silica shells, about 30 nm NiO nanoparticles were formed on the surface of the outer silica shell (FIGS. 1a and 1b). The amount of the NiO nanoparticles was enough to cover the whole surface of the outer silica shell. The high surface area of NiO particles exposed to outside allows interacting with high amount of the polyhistidine of protein.

The magnetic behavior of the magnetic nanocomposite was investigated by using a superconducting quantum interference device (SQUID) magnetometer (FIG. 1d). The field-dependent magnetization curve at 300 K reveals that the magnetic nanocomposite is slightly ferromagnetic with a high saturation magnetization of 142 emu/g, which is useful for repeated magnetic separation, and low coercivity. The magnetic nanocomposite was easily separated by using a permanent magnet from the aqueous solution and was readily redispersed in water by vortexing or sonification.

### Example 2

### Separation of His-Tagged GFP and UV Measurement

The magnetic nanocomposites were added to the solution of His-tagged GFP and/or untagged normal mouse IgG conjugated by PE-Cy5 in PBS (30 μg/ml, 200 μl), and incubated with vigorous shaking for 30 min to capture His-tagged protein. The magnetic nanocomposites were separated by magnet, redispersed into 0.1 g/ml imidazole solution and incubated with vigorous shaking for 30 min to release the captured protein. The magnetic nanocomposites in imidazole solution were retrieved by magnet to reuse, washed with PBS. Photoluminescent spectra were obtained by using Perkin-Elmer LS 50B. 400 nm and 630 nm excitation wavelengths were used for GFP and Cy5, respectively.

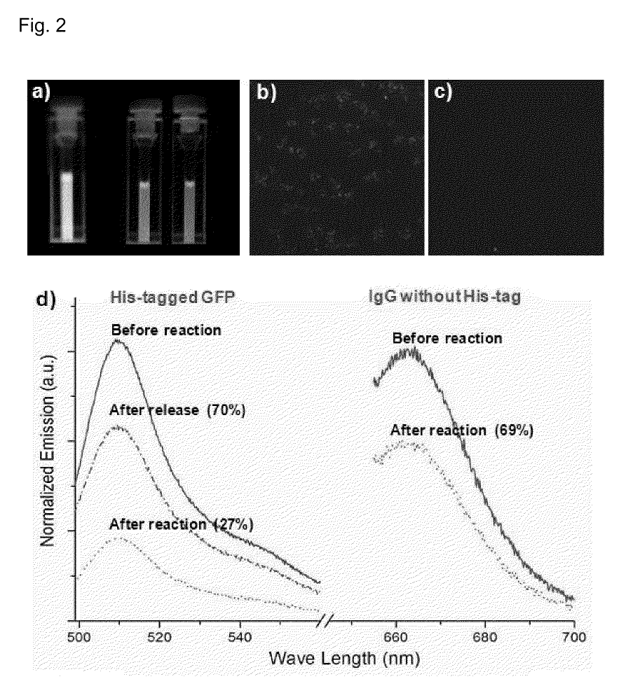

Firstly, this selective separation of His-tagged GFP from mixture of proteins was observed with the color of the solution under uv excitation. The original protein mixture of His-tagged GFP and untagged IgG-Cy5 was yellow colored (a left image in FIG. 2a). After incubation with the magnetic nanocomposite and magnetic separation of the magnetic composite/His-tagged GFP from the yellow colored mixture solution, the red colored supernatant was remained (a middle image in FIG. 2a). Finally, the released His-tagged GFP from the magnetic composite showed green-emitting solution (a right image in FIG. 2a).

In addition, the fluorescent image of the magnetic nanocomposite/His-tagged GFP after magnetic separation from the mixture protein solution showed strong green fluorescence (FIG. 2b) from the particles but negligible red-emission (FIG. 2c) was observed, verifying the selective attachment of His-tagged protein on the surface of NiO particles.

In order to quantify the protein separation efficacy, the fluorescent intensities of GFP and Cy5 before and after incubation with the magnetic nanocomposite and magnetic separation were measured (FIG. 2d). The fluorescent spectrum of the mixture of proteins before addition of the magnetic nanocomposite showed two maximum intensities at 510 nm and 663 nm, corresponding to GFP and Cy5 dye, respectively. After incubation with the magnetic nanocomposite, 73% decrease in intensity at 510 nm corresponding to the bound His-tagged GFP on the magnetic nanocomposite while only 31% decrease in intensity at 663 nm for the IgG without His-tag was resulted, indicating that the magnetic nanocomposite have superior binding property with His-tagged protein in comparison with untagged protein. After release of His-tagged GFP in imidazole solution, the fluorescent spectrum showed that 70% of the original His-tagged GFP was successfully separated from the mixture protein solution.

### Example 3

### Preparation of α-Synuclein and Cell Lysate of E. Coli Overexpressing His-Tagged α-Synuclein

The heat-treated cell lysate of E. coli overexpressing human α-synuclein was loaded onto DEAE-Sephacel anion-exchange column which was pre-equilibrated with 20 mM Tris-Cl, pH 7.5, containing 0.1 M NaCl, and eluted with 0.4 M NaCl under a linear gradient. Subsequently, Sephacryl S-200 size-exclusion chromatography was performed with 20 mM Mes, pH 6.5, containing 0.1% NaN₃. The α-synuclein-containing active fractions were combined and applied to S-Sepharose cation-exchange chromatography equilibrated with 20 mM Mes, pH 6.5. The purified α-synucleins were dialyzed against 20 mM Mes, pH 6.5, and stored at −20° C. in aliquots.

### Example 4

### Separation of His-Tagged α-Synuclein from E. Coil Lysate

2 mg of magnetic nanocomposite was added to 4 ml of E. coli lysates. After incubating for 30 min, the magnetic nanocomposite was separated by a magnet and washed with PBS twice to remove non-specifically absorbed lysates. In order to recover His-tagged α-synuclein from the magnetic nanocomposite, 0.1 g/ml imidazole solution was added and incubated for 30 min. The magnetic nanocomposite was retrieved with a magnet and washed with PBS to reuse. The recovered His-tagged α-synucleins were resolved on 12% SDS-PAGE gel. The gel was stained with coomassie blue.

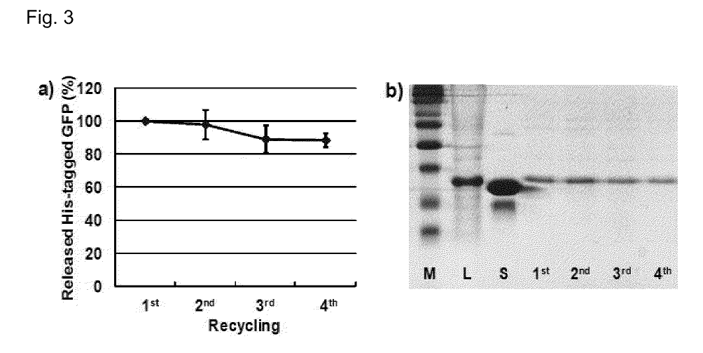

Each separated GFP amount compared to the initial separation capacity of the magnetic nanocomposite (FIG. 3a), representing that about 90% of selective separation capacity of His-tagged GFP from the mixture solution up to 4 times recycling. This is attributed to high magnetic moment of the magnetic nanocomposite since there might be negligible loss of the magnetic nanocomposite at each magnetic separation and washing step as well as to high surface area of histidine-binding NiO nanoparticles.

Even if the whole Ni cores are oxidized to NiO, the magnetic moment of the magnetic nanocomposite can be maintained because of the magnetite core inside the silica shell. This indicates that the combination of magnetic part with high magnetic moment and protein-binding NiO nanoparticles successfully worked in separation of His-tagged protein through magnetic recycling.

The SDS-PAGE analysis showed that His-tagged α-synucleins were well-separated by the magnetic nanocomposite up to 4 times (FIG. 3b). The magnetic nanocomposite showed good selectivity to His-tagged α-synuclein in the E. coli lysate and the binding property of the magnetic nanocomposite with His-tagged protein were maintained during several recycling steps.

What is claimed is:

1. A magnetic nanocomposite comprising a magnetic nanoparticle core of a magnetic nanoparticle, a silica shell coating said core, and a nanoparticle layer of a fourth period transition metal oxide, which coats said silica shell.

2. The magnetic nanocomposite of claim 1, wherein said magnetic nanoparticle is selected from the group consisting of transition metal oxides, transition metal phosphides, transition metal sulfides and transition metal alloys, and is ferromagnetic or superparamagnetic.

3. The magnetic nanocomposite of claim 2, wherein said transition metal is selected from the group consisting of iron (Fe), manganese (Mn), chromium (Cr), cobalt (Co), nickel (Ni), copper (Cu), zinc (Zn), samarium (Sm), gadolinium (Gd), neodymium (Nd), europium (Eu), barium (Ba) and platinum (Pt).

4. The magnetic nanocomposite of claim 1, wherein said magnetic nanoparticle core has a diameter ranging from 1 nm to 1,000 nm.

5. The magnetic nanocomposite of claim 1, wherein said silica shell is selected from the group consisting of crystalline silica, noncrystalline silica and porous silica.

6. The magnetic nanocomposite of claim 1, wherein said silica shell has a thickness ranging from 1 nm to 1,000 nm.

7. The magnetic nanocomposite of claim 1, wherein said fourth period transition metal is selected from the group consisting of chromium (Cr), manganese (Mn), iron (Fe), cobalt (Co), nickel (Ni), copper (Cu) and zinc (Zn).

8. The magnetic nanocomposite of claim 1, wherein said fourth period transition metal oxide has a diameter ranging from 1 nm to 100 nm.

9. The magnetic nanocomposite of claim 1, wherein said nanoparticle layer of a fourth period transition metal oxide has a thickness ranging from 1 nm to 1,000 nm.

10. A process for production of a magnetic nanocomposite, comprising:
(i) reacting a magnetic nanoparticle with a silica precursor to form a silica shell on said magnetic nanoparticle;
(ii) introducing a salt or ion of a fourth period transition metal to said silica shell; and
(iii) heating the particle formed in said step (ii) to form a nanoparticle layer of a fourth period transition metal oxide, which coats said silica shell.

11. The process of claim 10, wherein said magnetic nanoparticle is selected from the group consisting of transition metal oxides, transition metal phosphides, transition metal sulfides and transition metal alloys, and is ferromagnetic or superparamagnetic.

12. The process of claim 11, wherein said transition metal is selected from the group consisting of iron (Fe), manganese (Mn), chromium (Cr), cobalt (Co), nickel (Ni), copper (Cu), zinc (Zn), samarium (Sm), gadolinium (Gd), neodymium (Nd), europium (Eu), barium (Ba) and platinum (Pt).

13. The process of claim 10, wherein said magnetic nanoparticle core has a diameter ranging from 1 nm to 1,000 nm.

14. The process of claim 10, wherein said silica precursor is selected from the group consisting of tetraethyl orthosilicate (Si(OC₂H₅)₄), tetramethyl orthosilicate (Si(OCH₃)₄) and silicon tetrachloride (SiCl₄).

15. The process of claim 10, wherein said silica shell formed in said step (i) has a thickness ranging from 1 nm to 1,000 nm.

16. The process of claim 10, wherein said fourth period transition metal of said step (ii) is selected from the group consisting of chromium (Cr), manganese (Mn), iron (Fe), cobalt (Co), nickel (Ni), copper (Cu) and zinc (Zn).

17. The process of claim 10, wherein said step (iii) is carried out under hydrogen and nitrogen atmosphere at temperature of 100° C. to 1,000° C. for 10 min to 12 hr.

18. The process of claim 10, wherein said fourth period transition metal oxide in said step (iii) has a diameter ranging from 1 nm to 100 nm.

19. The process of claim 10, wherein said nanoparticle layer of a fourth period transition metal oxide in said step (iii) has a thickness ranging from 1 nm to 1,000 nm.

20. A reusable protein-binding agent which comprises a magnetic nanocomposite comprising a magnetic nanoparticle core of a magnetic nanoparticle, a silica shell coating said core, and a nanoparticle layer of a fourth period transition metal oxide, which coats said silica shell, said reusable protein-binding agent selectively binding a protein including an amino acid selected from the group consisting of histidine, asparagine, arginine, cystine, glutamine, lysine, methionine, proline and tryptophan.

21. The reusable protein-binding agent of claim 20, wherein said magnetic nanoparticle is selected from the group consisting of transition metal oxides, transition metal phosphides, transition metal sulfides and transition metal alloys, and is ferromagnetic or superparamagnetic.

22. The reusable protein-binding agent of claim 21, wherein said transition metal is selected from the group consisting of iron (Fe), manganese (Mn), chromium (Cr), cobalt (Co), nickel (Ni), copper (Cu), zinc (Zn), samarium (Sm), gadolinium (Gd), neodymium (Nd), europium (Eu), barium (Ba) and platinum (Pt).

23. The reusable protein-binding agent of claim 20, wherein said magnetic nanoparticle core has a diameter ranging from 1 nm to 1,000 nm.

24. The reusable protein-binding agent of claim 20, wherein said silica shell is selected from the group consisting of crystalline silica, noncrystalline silica and porous silica.

25. The reusable protein-binding agent of claim 20, wherein said silica shell has a thickness ranging from 1 nm to 1,000 nm.

26. The reusable protein-binding agent of claim 20, wherein said fourth period transition metal is selected from the group consisting of chromium (Cr), manganese (Mn), iron (Fe), cobalt (Co), nickel (Ni), copper (Cu) and zinc (Zn).

27. The reusable protein-binding agent of claim 20, wherein said fourth period transition metal oxide has a diameter ranging from 1 nm to 100 nm.

28. The reusable protein-binding agent of claim 20, wherein said nanoparticle layer of a fourth period transition metal oxide has a thickness ranging from 1 nm to 1,000 nm.

29. A process for selective binding, separation and purification of a protein, comprising:
(i) binding a protein-binding agent comprising a magnetic nanocomposite comprising a magnetic nanoparticle core of a magnetic nanoparticle, a silica shell coating said core, and a nanoparticle layer of a fourth period transition metal oxide, which coats said silica shell, with said protein contained in a protein mixture solution or cell lysate;
(ii) separating said protein bound with said protein-binding agent from said protein mixture solution or cell lysate by an external magnetic field; and
(iii) isolating said protein bound with said separated protein-binding agent from said magnetic nanocomposite.

30. The process of claim 29, wherein said magnetic nanoparticle is selected from the group consisting of transition metal oxides, transition metal phosphides, transition metal sulfides and transition metal alloys, and is ferromagnetic or superparamagnetic.

31. The process of claim 30, wherein said transition metal is selected from the group consisting of iron (Fe), manganese (Mn), chromium (Cr), cobalt (Co), nickel (Ni), copper (Cu), zinc (Zn), samarium (Sm), gadolinium (Gd), neodymium (Nd), europium (Eu), barium (Ba) and platinum (Pt).

32. The process of claim 29, wherein said magnetic nanoparticle core has a diameter ranging from 1 nm to 1,000 nm.

33. The process of claim 29, wherein said silica shell is selected from the group consisting of crystalline silica, noncrystalline silica and porous silica.

34. The process of claim 29, wherein said silica shell has a thickness ranging from 1 nm to 1,000 nm.

35. The process of claim 29, wherein said fourth period transition metal is selected from the group consisting of chromium (Cr), manganese (Mn), iron (Fe), cobalt (Co), nickel (Ni), copper (Cu) and zinc (Zn).

36. The process of claim 29, wherein said fourth period transition metal oxide has a diameter ranging from 1 nm to 100 nm.

37. The process of claim 29, wherein said nanoparticle layer of a fourth period transition metal oxide has a thickness ranging from 1 nm to 1,000 nm.

38. The process of claim 29, wherein said protein is a protein including at least one amino acid selected from the group consisting of histidine, asparagine, arginine, cystine, glutamine, lysine, methionine, proline and tryptophan.

39. The process of claim 29, wherein said protein is a protein including histidine.
